SEQUENCE LISTING (8 sequences)

SEQ ID NO: 1 - protein, 503 aa, Vigna angularis
MAQVQVQPQNAMPGPNGAAAAAGGNQFVTTSLYVGDLDPNVTDSQLYDLFSQLGQVVSVRVCRDLTSRRSLGYGYVNYSNPQDAARALDVLNFTPLNNKPIRIMYSHRDPCIRKSGAGNIFIKNLDRAIDHKALHDTFSTFGNILSCKVATDSSGQSKGYGFVQFDNEESAQKAIEKLNGMLLNDKQVYVGPFLRKQERETAIDKAKFNNVFVKNLADSTSDDELKTIFGEFGTITSAVVMRDGDGKSKCFGFVNFENADDAARAVEALNGKKFDDKEWYVGKAQKKSERENELKQRFEQSMKEAADKYQGANLYVKNLDDSISDDKLKELFSPFGTITSCKVMRDPNGVSRGSGFVAFSTPEEASRALSEMNGKMVVSKPLYVTLAQRKEDRRARLQAQFAQMRPVGMPPSVGPRVPMYPPGGPGIGQQIFYGQGPPAIIPSQAGFGYQQQLVPGMRPGAAPVPNFFVPMVQQGQQGQRPGGRRAVQQSQQPVPMMPQQMLP

SEQ ID NO: 2 - DNA, 1511 bp, Artificial Sequence
  cDNA
atggctcaggttcaggttcagcctcagaatgcgatgcccggtcccaacggtgctgctgctgctgctgggggaaaccagttcgttacgacatcgctttacgtcggagatctcgaccccaacgtcacggactcacagctttatgacctgttcagtcaattgggccaagttgtgtctgttagggtttgcagggacttgaccagccgaagatcgctcggttacggctatgtcaactatagcaacccccaagatgctgccagagcattagatgttctgaatttcactcctctcaacaacaagcccatccgaattatgtattcacatcgtgatccctgtatccggaaaagtggggcaggaaatatttttatcaagaatttggatagggcaattgaccacaaggcattacatgataccttctctacatttgggaatatcctttcatgcaaggtagcaacggattcatctgggcaatcaaaaggatatggttttgttcagtttgataatgaggaatctgcccaaaaagccatagagaagctgaatggtatgctgttgaatgataagcaagtgtatgtgggacccttccttcgcaagcaagagagagagactgctattgacaaggcaaaattcaataatgtttttgtaaagaatctagcagattcgactagtgatgatgaattgaagacaatttttggtgaatttggaactattactagtgctgtagtgatgagggatggagatgggaaatcaaagtgctttgggtttgtgaattttgagaatgctgatgatgctgctagggctgttgaggctctcaatggcaaaaaatttgatgataaggaatggtacgttggaaaagctcagaagaaatctgaaagggagaatgaattgaaacaacgatttgagcagagcatgaaagaagctgctgataaatatcaaggggcaaacttgtatgtcaaaaatttggatgatagcattagtgatgataaacttaaggagctgttctccccttttggtaccatcacctcttgcaaggttatgagggacccaaatggcgttagtcgtggatctggatttgttgcattctcaactcctgaggaggcatctagagcactctctgagatgaatgggaaaatggtggtaagtaaacctctgtatgtgactctagcccaaaggaaagaagatagaagagctagactgcaggctcagtttgctcaaatgcgacctgttggaatgccaccatctgttggtcctcgtgtgccaatgtatcctccaggtggtccaggtattggtcaacaaatattttatggccaaggccctcctgctatcattccttcccaggccggatttggttaccaacaacaacttgtgcctggtatgaggccaggtgcagctcctgtgccaaatttctttgtgccaatggttcagcagggacaacagggccagcgccctggtggaaggcgtgcagtccagcagtcccagcagccagttccaatgatgccacagcagatgcttcctag

SEQ ID NO: 3 - DNA, 36 bp, Artificial Sequence
  forward primer
cgacgacaagaccctatggctcaggttcaggttcag

SEQ ID NO: 4 - DNA, 36 bp, Artificial Sequence
  reverse primer
gaggagaagagcccctaggaagcatctgctgtggca

SEQ ID NO: 5 - DNA, 18 bp, Artificial Sequence
  forward primer
cgctcggttacggctatg

SEQ ID NO: 6 - DNA, 18 bp, Artificial Sequence
  reverse primer
gcttgcgaaggaagggtc

SEQ ID NO: 7 - DNA, 19 bp, Artificial Sequence
  forward primer
ccctcccacatgctattct

SEQ ID NO: 8 - DNA, 19 bp, Artificial Sequence
  reverse primer
agagcctccaatccagaca

(19) United States
(12) Patent Application Publication
(10) Pub. No.: US 2024/0043858 A1
(43) Pub. Date: Feb. 8, 2024

(54) A Protein Vapbp2-L For Enhancing Drought Resistance Of Plants And Application Thereof

(71) Applicant: Hainan University, Haikou City, Hainan Province (CN)

(72) Inventors: Aihua SHA, Jingzhou, Hubei Province (CN); Yinhua CHEN, Haikou City, Hainan Province (CN); Yanjuan WANG, Jingzhou, Hubei Province (CN); Haozhong JIANG, Jingzhou, Hubei Province (CN); Lintao HUANG, Jingzhou, Hubei Province (CN); Yantao XIANG, Jingzhou, Hubei Province (CN); Zhengxin WEI, Jingzhou, Hubei Province (CN)

(73) Assignee: Hainan University, Haikou City, Hainan Province (CN)

(21) Appl. No.: 17/624,425

(22) PCT Filed: Dec. 30, 2021
(86) PCT No.: PCT/CN2021/143327

§ 371 Date: Jan. 3, 2022

(30) Foreign Application Priority Data
Nov. 1, 2021 (CN) 202111280421.0

(51) Int. Cl. C12N 15/82; C07K 14/415

(52) U.S. Cl. CPC C12N 15/8273; C12N 15/8205; C07K 14/415

(57) ABSTRACT

The present invention provides a protein VaPBP2-L that enhances plant drought resistance and its coding gene and application. The protein VaPBP2-L gene is used to construct various plant expression vectors, which can be widely used in the cultivation of new drought-resistant varieties of transgenic plants and crop, and can be used for drought-resistant breeding of plants as the drought-resistant genetic resources, and promotes the breeding process of new drought-resistant varieties (lines) of crops and plant.

### TECHNICAL FIELD

The present invention relates to the technical field of biological genetic engineering, in particular to a protein VaPBP2-L that enhances plant drought resistance and its coding gene and application.

### BACKGROUND ARTS

With the increasing global warming in recent years, drought has become one of the main abiotic stresses that cause crop yield reduction. In China, food losses due to drought accounted for more than 50% of those caused by all natural disasters. Therefore, it is a significant challenge for solving the drought problem to achieve sustainable agricultural development.

Drought-resistant breeding of traditional crops is restricted by such factors as long drought-resistant breeding cycle, high investment and limited germplasm resources, resulting in slow progress of drought-resistant breeding at present. Biotechnology breeding can break the restriction between species and provide a new way of efficient drought-resistant breeding. Therefore, identification and screening of drought-resistant gene resources is the key to obtain new varieties of drought-resistant transgenic crops. At present, the research on drought resistance of adzuki bean (Vigna angularis L.) is progressing slowly, which mainly identifies and screens drought-resistant adzuki bean by using the morphological index and physiological and biochemical measurement of adzuki bean seedlings as the index for evaluating the drought resistance of adzuki bean. For example, Jianjun HAO took adzuki bean as research materials and measured physiological indexes such as peroxidase and electrical conductivity during the growth of adzuki bean as drought-resistant indexes to screen out drought-resistant varieties. However, there are few researches at present on searching for new drought-resistant genes while screening the drought-resistant germplasm resources of adzola bean, and using them as effective drought-resistant gene resources for drought-resistant breeding of other crops by mining and analyzing drought-resistant genes. Therefore, when screening and identifying the drought-resistant seed resources of adzuki bean, it is necessary to effectively obtain and identify its related resistance protein genes so as to provide drought-resistant gene resources for crop drought-resistant breeding.

### SUMMARY OF INVENTION

In view of this, the present invention provides a protein VaPBP2-L that enhances plant drought resistance and its coding gene and application. The present invention uses adzuki bean germination seeds as materials to isolate the VaPBP2-L gene, constructs a virus expression vector from the gene, and transforms it into tobacco later, which can significantly improve the drought tolerance of plants. The gene of protein VaPBP2-L is used to construct various plant expression vectors, which can be widely used in the cultivation of transgenic plants and new drought-resistant varieties of crops.

The technical solutions of the present invention are as follows.

A protein VaPBP2-L that enhances the drought resistance of plants is provided, wherein the protein VaPBP2-L is derived from Adzuki bean (Vigna angularis L.), and its amino acid sequence is shown in SEQ ID NO: 1.

Further, the nucleotide sequence of the gene encoding protein VaPBP2-L is shown in SEQ ID NO: 2.

Further, the specific primers for PCR amplification of the gene encoding protein VaPBP2-L are:

VaPBP2-L-F1:
(SEQ ID NO: 3)
5′-CGACGACAAGACCCTATGGCTCAGGTTCAGGTTCAG-3′;
and
VaPBP2-L-R1:
(SEQ ID NO: 4)
5′-GAGGAGAAGAGCCCCTGGAAGCATCTGCTGTGGCA-3′

Further, the recombinant expression vector for expressing the protein VaPBP2-L is obtained by inserting the target gene between the LIC1 and LIC2 sites of the vector PVX-LIC.

Embodiments of the present invention also provide application of a protein VaPBP2-L for enhancing the drought resistance of plants.

Embodiments of the present invention also provide application of a protein VaPBP2-L that enhances the drought resistance of plants for regulating the drought resistance of tobacco.

Embodiments of the present invention also provide a recombinant protein VaPBP2-L that enhances drought resistance of plants. The recombinant protein VaPBP2-L is derived from adzuki bean (Vigna angularis L.).

In some embodiments, the amino acid sequence of the recombinant protein VaPBP2-L is shown in SEQ ID NO: 1.

In some embodiments, the plant is not Adzuki bean (Vigna angularis L.).

In some embodiments, the plant is tobacco.

Embodiments of the present invention also provide a nucleic acid encoding the recombinant protein VaPBP2-L, wherein the nucleic acid has a sequence as shown in SEQ ID NO: 2.

Embodiments of the also provide a cDNA encoding a protein VaPBP2-L, wherein the amino acid sequence of the protein VaPBP2-L is shown in SEQ ID NO: 1.

In some embodiments, the cDNA has the sequence shown in SEQ ID NO: 2.

Embodiments of the present invention also provide a recombinant expression vector, which comprises the aforementioned nucleic acid or cDNA.

In some embodiments, the recombinant expression vector is a recombinant PVX-LIC vector, which is formed by inserting the nucleic acid or the cDNA between the LIC1 and LIC2 sites of the PVX-LIC vector.

Embodiments of the present invention also provide an Agrobacterium tumefaciens, which comprises any one of the aforementioned recombinant expression vectors.

In some embodiments, the Agrobacterium tumefaciens is Agrobacterium tumefaciens GV3101.

Embodiments of the present invention also provide a method for enhancing drought resistance of plants, which comprises transforming plants with the aforementioned recombinant expression vector or the aforementioned Agrobacterium tumefaciens.

Embodiments of the present invention also provide application of the aforementioned nucleic acid encoding the recombinant protein VaPBP2-L for enhancing the drought resistance of plants.

Embodiments of the present invention also provide application of the aforementioned cDNA encoding the protein VaPBP2-L for enhancing the drought resistance of plants.

Unless otherwise specified, the terms “a gene encoding (the) protein VaPBP2-L” and “a nucleic acid encoding (the) protein VaPBP2-L” have the same meaning in the present invention.

Compared with the prior art, the present invention finds out an extremely drought-tolerant adzuki bean germplasm by identifying the drought-resistant germplasm resources of adzuki bean, and identifies the drought resistance protein VaPBP2-L by employing extremely drought-resistant and extremely sensitive adzuki bean germplasm as materials, and by using proteomic sequencing method to analyze the difference in protein accumulation between drought resistance germplasm and sensitive germplasm under drought stress conditions. In addition, the gene encoding VaPBP2-L is cloned from drought-resistant adzuki bean varieties, and overexpressed through a viral expression vector in tobacco. The drought resistance of tobacco was significantly improved. The drought resistance function of the protein VaPBP2-L was quickly identified, which confirmed that the protein VaPBP2-L can improve the drought tolerance of plants and thus can be effectively used as a drought-resistant gene resource for drought-resistant breeding of plants, and promote the breeding process of drought-resistant new varieties (lines) of crops and plant.

### BRIEF DESCRIPTION OF THE DRAWINGS

FIG. 1 is the result of amplification of the VaPBP2-L gene cDNA coding nucleotide sequence in the embodiment of the present invention, wherein M is D2000 Plus Marker, and the size of the bands from top to bottom is 5000, 3000, 2000, 1000, 750, 500, 250, and 100 bp;

FIG. 2 shows the PCR identification of Agrobacterium introduced with the recombinant plasmid PVX-LIC-VaPBP2-L in the embodiment of the present invention, wherein 1-7 are monoclonal numbers, H₂O is a blank control, and “M” is a Marker;

FIG. 3 shows the expression of VaPBP2-L in tobacco plants overexpressing VaPBP2-L by viral expression vector detected by RT-PCR in an embodiment of the present invention, wherein M is DL2000 Plus marker, lanes 1-4 are uninjected tobacco under normal growth, uninjected tobacco under drought treatment, tobacco transformed with PVX-LIC empty vector, and tobacco transformed with PVX-LIC-VaPBP2-L plasmid separately;

FIG. 4 shows the phenotype of tobacco overexpressing VaPBP2-L under drought stress in an embodiment of the present invention. In the figure, A shows uninjected tobacco before drought treatment (0 d), uninjected tobacco, tobacco transformed with PVX-LIC empty vector, and tobacco transformed with PVX-LIC-VaPBP2-L plasmid from left to right, and B shows the uninjected tobacco for 15 days of normal-growth, the uninjected tobacco for 15 days of drought treatment, the tobacco transformed with PVX-LIC empty vector for 15 days of drought treatment, and the tobacco transformed with PVX-LIC-VaPBP2-L plasmid for 15 days from left to right.

### DETAILED EMBODIMENTS

The present invention will be understood more readily by reference to the detailed description of the following examples.

Unless otherwise specified, the experimental methods used in the examples of the present invention are all conventional methods.

Unless otherwise specified, the materials, reagents, etc. used in the examples of the present invention can be obtained from commercial sources.

### Example 1—the Acquisition of Protein VaPBP2-L and its Coding Gene and Recombinant Expression Vector

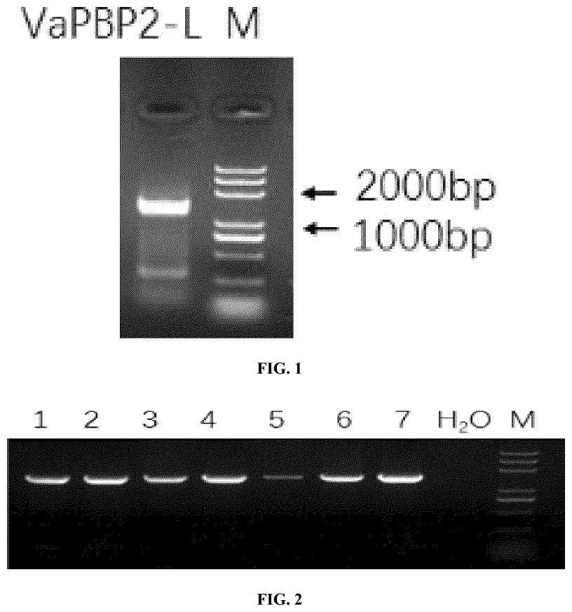

(1) Adzuki bean seeds treated with 9.0% mannitol stress for 36 h were used as materials to extract total RNA and to obtain cDNA by reverse transcription. Amplification was performed by a conventional PCR method, using the cDNA as a template, under the guidance of primer VaPBP2-L-F1 (SEQ ID NO: 3) and primer VaPBP2-L-R1 (SEQ ID NO: 4). After the reaction, the PCR amplified product was detected by 1% agarose gel electrophoresis, and the DNA fragment of about 1511 bp was recovered and purified, as shown in FIG. 1.

(2) The gene fragment was ligated to the vector PVX-LIC (with a lethal gene ccdB on the T-DNA fragment of the vector PVX-LIC, and with the recognition sequence for the LIC reaction on both flank of the lethal gene ccdB. The vector is owned by own laboratory. Literature: Zhao J, Liu Q, Hu P, et al (2016) An efficient Potato virus X-based microRNA silencing in Nicotiana benthamiana. Sci Rep 6: 20573) by using LIC (ligation—independent cloning) reaction. The recombinant vector PVX-LIC-VaPBP2-L was obtained. The sequencing confirmed that the recombinant vector PVX-LIC-VaPBP2-L was such that the target gene (that is, the 1511 bp DNA fragment shown in SEQ ID NO: 2) was inserted between the LIC1 and LIC2 sites of the vector PVX-LIC, as shown in FIG. 2. The coding gene with the nucleotide sequence of SEQ ID NO: 2 is named VaPBP2-L, which encodes the protein VaPBP2-L shown in SEQ ID NO: 1 consisting of 503 amino acids.

The sequence of the above PCR amplification primers is as follows:

VaPBP2-L-F1:
(SEQ ID NO: 3)
5′-CGACGACAAGACCCTATGGCTCAGGTTCAGGTTCAG-3′;
and
VaPBP2-L-R1:
(SEQ ID NO: 4)
5′-GAGGAGAAGAGCCCCTGGAAGCATCTGCTGTGGCA-3′.

The amino acid sequence of protein VaPBP2-L (SEQ ID NO: 1) is as follows, consisting of 503 amino acids:

| | |
|---|---|
| 1 | MAQVQVQPQN AMPGPNGAAA AAGGNQFVTT SLYVGDLDPN |
| | VTDSQLYDLF SQLGQVVSVR VCRDLTSRRS LGYGYVNYSN |
| 81 | PQDAARALDV LNFTPLNNKP IRIMYSHRDP CIRKSGAGNI FIKNLDRAID |
| | HKALHDTFST FGNILSCKVA TDSSGQSKGY |
| 161 | GFVQFDNEES AQKAIEKLNG MLLNDKQVYV GPFLRKQERE |
| | TAIDKAKFNN VFVKNLADST SDDELKTIFG EFGTITSAVV |
| 241 | MRDGDGKSKC FGFVNFENAD DAARAVEALN GKKFDDKEWY |
| | VGKAQKKSER ENELKQRFEQ SMKEAADKYQ GANLYVKNLD |
| 321 | DSISDDKLKE LFSPFGTITS CKVMRDPNGV SRGSGFVAFS TPEEASRALS |
| | EMNGKMVVSK PLYVTLAQRK EDRRARLQAQ |
| 401 | FAQMRPVGMP PSVGPRVPMY PPGGPGIGQQ IFYGQGPPAI |
| | IPSQAGFGYQ QQLVPGMRPG AAPVPNFFVP MVQQGQQGQR |
| 481 | PGGRRAVQQS QQPVPMMPQQ MLP |

The protein VaPBP2-L gene cDNA coding nucleotide sequence (SEQ ID NO: 2) is as follows, with a length of 1511 bp:

| | |
|---|---|
| 1 | ATGGCTCAGG TTCAGGTTCA GCCTCAGAAT GCGATGCCCG |
| | GTCCCAACGG TGCTGCTGCT GCTGCTGGGG GAAACCAGTT |
| 81 | CGTTACGACA TCGCTTTACG TCGGAGATCT CGACCCCAAC |
| | GTCACGGACT CACAGCTTTA TGACCTGTTC AGTCAATTGG |
| 161 | GCCAAGTTGT GTCTGTTAGG GTTTGCAGGG ACTTGACCAG |
| | CCGAAGATCG CTCGGTTACG GCTATGTCAA CTATAGCAAC |
| 241 | CCCCAAGATG CTGCCAGAGC ATTAGATGTT CTGAATTTCA |
| | CTCCTCTCAA CAACAAGCCC ATCCGAATTA TGTATTCACA |
| 321 | TCGTGATCCC TGTATCCGGA AAAGTGGGGC AGGAAATATT |
| | TTTATCAAGA ATTTGGATAG GGCAATTGAC CACAAGGCAT |
| 401 | TACATGATAC CTTCTCTACA TTTGGGAATA TCCTTTCATG |
| | CAAGGTAGCA ACGGATTCAT CTGGGCAATC AAAAGGATAT |
| 481 | GGTTTTGTTC AGTTTGATAA TGAGGAATCT GCCCAAAAAG |
| | CCATAGAGAA GCTGAATGGT ATGCTGTTGA ATGATAAGCA |
| 561 | AGTGTATGTG GGACCCTTCC TTCGCAAGCA AGAGAGAGAG |
| | ACTGCTATTG ACAAGGCAAA ATTCAATAAT GTTTTTGTAA |
| 641 | AGAATCTAGC AGATTCGACT AGTGATGATG AATTGAAGAC |
| | AATTTTTGGT GAATTTGGAA CTATTACTAG TGCTGTAGTG |
| 721 | ATGAGGGATG GAGATGGGAA ATCAAAGTGC TTTGGGTTTG |
| | TGAATTTTGA GAATGCTGAT GATGCTGCTA GGGCTGTTGA |
| 801 | GGCTCTCAAT GGCAAAAAAT TTGATGATAA GGAATGGTAC |
| | GTTGGAAAAG CTCAGAAGAA ATCTGAAAGG GAGAATGAAT |
| 881 | TGAAACAACG ATTTGAGCAG AGCATGAAAG AAGCTGCTGA |
| | TAAATATCAA GGGGCAAACT TGTATGTCAA AAATTTGGAT |
| 961 | GATAGCATTA GTGATGATAA ACTTAAGGAG CTGTTCTCCC |
| | CTTTTGGTAC CATCACCTCT TGCAAGGTTA TGAGGGACCC |
| 1041 | AAATGGCGTT AGTCGTGGAT CTGGATTTGT TGCATTCTCA |
| | ACTCCTGAGG AGGCATCTAG AGCACTCTCT GAGATGAATG |
| 1121 | GGAAAATGGT GGTAAGTAAA CCTCTGTATG TGACTCTAGC |
| | CCAAAGGAAA GAAGATAGAA GAGCTAGACT GCAGGCTCAG |
| 1201 | TTTGCTCAAA TGCGACCTGT TGGAATGCCA CCATCTGTTG |
| | GTCCTCGTGT GCCAATGTAT CCTCCAGGTG GTCCAGGTAT |
| 1281 | TGGTCAACAA ATATTTTATG GCCAAGGCCC TCCTGCTATC |
| | ATTCCTTCCC AGGCCGGATT TGGTTACCAA CAACAACTTG |
| 1361 | TGCCTGGTAT GAGGCCAGGT GCAGCTCCTG TGCCAAATTT |
| | CTTTGTGCCA ATGGTTCAGC AGGGACAACA GGGCCAGCGC |
| 1441 | CCTGGTGGAA GGCGTGCAGT CCAGCAGTCC CAGCAGCCAG |
| | TTCCAATGAT GCCACAGCAG ATGCTTCCTA G |

### Example 2—Acquisition of Recombinant Agrobacterium tumefaciens

The recombinant vector PVX-LIC-VaPBP2-L was transformed into Agrobacterium tumefaciens GV3101 by a freeze-thaw method to obtain Agrobacterium tumefaciens GV3101 containing the recombinant vector PVX-LIC-VaPBP2-L. The recombinant Agrobacterium was named GV3101/PV X-LIC-VaPBP2-L; (The freeze-thaw method is referred to Amanda M Davis, Anthony Hall, Andrew J Millar, Chiarina Darrah and Seth J Davis, Protocol: Streamlined sub-protocols for floral-dip transformation and selection of transformants in Arabidopsis thaliana, 2009, publicly available from Yangtze University).

The empty vector PVX-LIC was transformed into Agrobacterium tumefaciens GV3101 by the freeze-thaw method to obtain Agrobacterium tumefaciens GV3101 containing the empty vector PVX-LIC, and the recombinant Agrobacterium was named GV3101/PVX-LIC.

### Example 3—Acquisition and Identification of Transiently Expressing Transgenic Tobacco

(1) Acquisition of Transgenic Tobacco

The two recombinant Agrobacterium GV3101/PVX-LIC-VaPBP2-L and GV3101/PVX-LIC obtained in Example 2 were used to prepare an Agrobacterium suspension, and the volume ratio of the culture solution to the bacteria in the suspension was 1:1. Tobacco (Nicotiana benthamiana) seeds were sown in a culture medium (a mixture of peat:vermiculite:perlite in a volume ratio of 1:3:0.5) and cultivated in an artificial greenhouse. When the tobacco grew to 4-5 leaves, injection was started on the new leaves fully expanded at the top. 1 mL of bacterial solution was drawn by a disposable syringe respectively, and then the needle of the syringe was removed. The lower part of the leaf was press with fingers, and the bacteria solution in the syringe was gently forced to penetrate into the leaf tissue. 2 leaves of each tobacco plant were injected, and GV3101/PVX-LIC-VaPBP2-L and GV3101/PVX-LIC were injected to 5 plants respectively.

The injected tobacco plants were covered with a plastic film and cultured for 24 hours in the dark, and then moved to a greenhouse at 25° C. under a photoperiod of 16 hours of light/8 hours of darkness. Tobacco without Agrobacterium injection was used as a wild-type control and cultured under the same growth conditions to obtain VaPBP2-L positive transgenic plants, plants transformed with empty vector and wild-type plants, respectively.
(2) Molecular Detection of Transgenic Tobacco

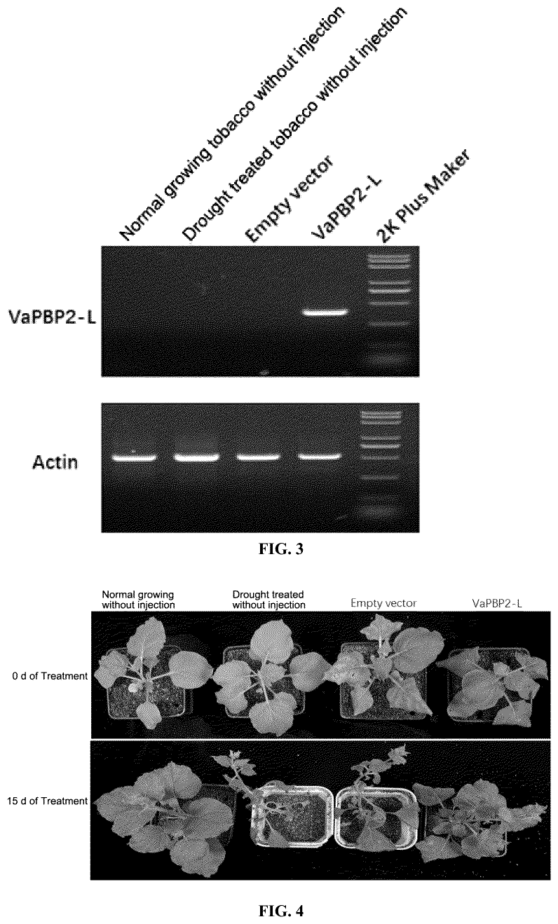

The VaPBP2-L positive transgenic plant, empty vector plant and wild-type plant obtained in step (1) were employed to extract total RNA respectively and to obtain cDNA by reverse transcription. RT-PCR amplification was performed by using the cDNA as a template, and using the specific primer VaVPAC-F2 5′-CGCTCGGTTACGGCTATG-3′ (SEQ ID NO: 5) and the reverse primer VaVPAC-R2 5′-GCTTGCGAAGGAAGGGTC-3′ (SEQ ID NO: 6). The tobacco actin was used as an internal reference, using primers FC 5′-CCCTCCCACATGCTATTCT-3′ (SEQ ID NO: 7), and RC 5′-AGAGCCTCCAATCCAGACA-3′ (SEQ ID NO: 8). The results are shown in FIG. 3, indicating that the target gene VaPBP2-L was not expressed in the plant transformed with vector and wild-type plant; while the target gene VaPBP2-L was expressed in the transgenic VaPBP2-L plant, indicating that the transgenic tobacco strain with transient expression of VaPBP2-L was obtained.

(3) Drought Resistance Phenotype Identification of Transgenic Tobacco

The VaPBP2-L positive transgenic tobacco strain, tobacco strain transformed with empty vector and wild-type strain obtained in step (1) were subjected to a drought stress treatment 7 days after the injection. At 15 days (soil water content dropped to 7.16%), uninjected wild-type plants and empty vector control plants (empty vector) could be observed for severe wilting, while the tobacco injected with PVX-LIC-VaPBP2-L gene exhibits good drought resistance. The drought resistance under drought conditions of tobacco injected with PVX-LIC-VaPBP2-L gene was significantly stronger than that of wild-type and empty vector expression tobacco, which was similar to the growth effect of the group of uninjected tobacco under normal growth (without drought treatment), as shown in FIG. 4. This indicates that the VaPBP2-L gene can significantly improve the drought resistance of tobacco, and this gene can be used for drought resistance breeding of plants or crops.

The above mentioned are only preferred embodiments of the present invention and are not intended to limit the present invention. Any modification, equivalent replacement, improvement etc. made within the spirit and principle of the present invention shall be included in the scope of protection of the present invention.

1. A protein VaPBP2-L for enhancing drought resistance of plants, wherein the protein VaPBP2-L is derived from Adzuki bean (Vigna angularis L.), and its amino acid sequence is shown in SEQ ID NO: 1.

2. A coding gene of the protein VaPBP2-L for enhancing drought resistance of plants of claim 1, wherein the coding gene has a nucleotide sequence shown in SEQ ID NO: 2.

3. A coding gene of the protein VaPBP2-L for enhancing drought resistance of plants of claim 1, wherein the specific primers for PCR amplification of the coding gene of the protein VaPBP2-L are:
VaPBP2-L-F1:
(SEQ ID NO: 3)
5′-CGACGACAAGACCCTATGGCTCAGGTTCAGGTTCAG-3′;
and
VaPBP2-L-R1:
(SEQ ID NO: 4)
5′-GAGGAGAAGAGCCCCTGGAAGCATCTGCTGTGGCA-3′

4. A coding gene of the protein VaPBP2-L for enhancing drought resistance of plants of claim 1, wherein a recombinant expression vector is obtained by inserting a coding gene of the protein VaPBP2-L between the LIC1 and LIC2 sites of a vector PVX-LIC.

5. Application of the protein VaPBP2-L for enhancing drought resistance of plants of claim 1.

6. The application of claim 5, wherein the protein VaPBP2-L is used for regulating the drought resistance of tobacco.

7. A recombinant protein VaPBP2-L for enhancing drought resistance of plants, wherein the recombinant protein VaPBP2-L is derived from adzuki bean (Vigna angularis L.) and has an amino acid sequence shown in SEQ ID NO: 1.

8. A nucleic acid coding the recombinant protein VaPBP2-L of claim 7.

9. The nucleic acid of claim 8, wherein the nucleic acid has a sequence as shown in SEQ ID NO: 2.

10. A recombinant expression vector, comprising the nucleic acid of claim 8.

11. The recombinant expression vector of claim 10, which is a recombinant PVX-LIC vector and formed by inserting the nucleic acid between the LIC1 and LIC2 sites of a PVX-LIC vector.

12. An Agrobacterium tumefaciens, comprising the recombinant expression vector of claim 10.

13. The Agrobacterium tumefaciens according to claim 12, which is Agrobacterium tumefaciens GV3101.

14. A method for enhancing drought resistance of plants, comprising transforming plants with the recombinant expression vector of claim 10.

15. A recombinant expression vector, comprising the nucleic acid of claim 9.

16. The recombinant expression vector of claim 15, which is a recombinant PVX-LIC vector and formed by inserting the nucleic acid between the LIC1 and LIC2 sites of a PVX-LIC vector.

17. An Agrobacterium tumefaciens, comprising the recombinant expression vector of claim 11.

18. An Agrobacterium tumefaciens, comprising the recombinant expression vector of claim 15.

19. An Agrobacterium tumefaciens, comprising the recombinant expression vector of claim 16.

20. A method for enhancing drought resistance of plants, comprising transforming plants with the Agrobacterium tumefaciens of claim 12.

21. Application of the coding gene of claim 2 in enhancing the drought resistance of plants.

22. The application of claim 21, wherein the coding gene is used for regulating the drought resistance of tobacco.
